(19) United States
(12) Patent Application Publication
(10) Pub. No.: US 2019/0250140 A1
(43) Pub. Date: Aug. 15, 2019

(54) BIOLOGICAL DETECTION CHIP AND DETECTING METHOD THEREOF

(71) Applicants: BEIJING BOE OPTOELECTRONICS TECHNOLOGY CO., LTD., Beijing (CN); BOE TECHNOLOGY GROUP CO., LTD., Beijing (CN)

(72) Inventors: Le GU, Beijing (CN); Peizhi CAI, Beijing (CN); Fengchun PANG, Beijing (CN); Yue GENG, Beijing (CN); Chuncheng CHE, Beijing (CN)

(21) Appl. No.: 16/154,011

(22) Filed: Oct. 8, 2018

(30) Foreign Application Priority Data
Feb. 14, 2018 (CN) 201810151878.3

(51) Int. Cl. G01N 33/48; G01N 21/59; G02B 6/122; G01N 21/63

(52) U.S. Cl. CPC G01N 33/48; G01N 21/63; G02B 6/122; G01N 21/59

(57) ABSTRACT

The present disclosure relates to a biological detection chip and a detecting method thereof. The biological detection chip includes a light guiding substrate having a top surface, a bottom surface opposite to the top surface, and a side surface between the top surface and the bottom surface, and a biosensitive membrane on the top surface of the light guiding substrate.

### CROSS REFERENCE TO RELATED APPLICATIONS

This patent application claims the benefit and priority of Chinese Patent Application No. 201810151878.3 filed on Feb. 14, 2018, the disclosure of which is incorporated by reference herein in its entirety as part of the present application.

### BACKGROUND

Embodiments of the present disclosure relate to a field of displaying technologies, and in particular, to a biological detection chip and a detecting method thereof.

The biological detection chip mostly uses fluorescent or other methods to label the objective molecule, and detects the optical signal and the electrical signal through a specific reaction between the objective molecule and the target molecule immobilized on the biological chip. Most of the current detecting methods are fluorescence detection, chemiluminescence detection, and electrochemical detection. In addition to involving complex fluorescent labels, optical detection systems include electrode circuits, resulting in complex preprocessing steps of sample, cumbersome operation, and high detection costs.

### BRIEF DESCRIPTION

Embodiments of the present disclosure provide a biological detection chip and a detecting method thereof.

A first aspect of the present disclosure provides a biological detection chip. The biological detection chip includes a light guiding substrate having a top surface, a bottom surface opposite to the top surface, and a side surface between the top surface and the bottom surface, and a biosensitive membrane on the top surface of the light guiding substrate.

In an embodiment of the present disclosure, the biological detection chip further includes a first optical confinement layer on the top surface of the light guiding substrate and a second optical confinement layer on the bottom surface of the light guiding substrate, wherein the first optical confinement layer has an opening exposing the light guiding substrate, and the biosensitive membrane is located within the opening.

In an embodiment of the present disclosure, one of the first optical confinement layer and the second optical confinement layer includes a light transmissive material, and the other one of the first optical confinement layer and the second optical confinement layer includes a reflective material or an opaque material, wherein a refractive index of the light transmissive material is smaller than a refractive index of the light guiding substrate.

In an embodiment of the present disclosure, the first optical confinement layer and the second optical confinement layer include a light transmissive material, and a refractive index of the light transmissive material is smaller than a refractive index of the light guiding substrate.

In an embodiment of the present disclosure, the first optical confinement layer and the second optical confinement layer include a reflective material or an opaque material.

In an embodiment of the present disclosure, a material of the biosensitive membrane includes one of an antibody probe, an enzyme probe, and a DNA probe.

In an embodiment of the present disclosure, a material of the light guiding substrate includes glass or resin.

A second aspect of the present disclosure provides a detecting method using the biological detection chip in the first aspect of the present disclosure. The detecting method includes before contacting a sample to be detected with the biosensitive membrane, introducing a light for detection from one end of the light guiding substrate and detecting an exit light from the other end of the light guiding substrate to obtain a reference signal characterizing the exit light, after contacting the sample to be detected with the biosensitive membrane, detecting the exit light to obtain a detection signal characterizing the exit light, and obtaining an information of an objective molecule corresponding to the biosensitive membrane in the sample to be detected based on a change of the detection signal with respect to the reference signal.

In an embodiment of the present disclosure, the reference signal and the detection signal include a light power.

In an embodiment of the present disclosure, the information includes a concentration of the objective molecule.

In an embodiment of the present disclosure, after obtaining the detection signal, obtaining the concentration of the objective molecule based on a dependency relationship between the detection signal obtained in advance and the concentration of the objective molecule.

In an embodiment of the present disclosure, the dependency relationship is determined by the following steps: detecting standard samples containing objective molecules of different known concentrations, respectively, by using a plurality of standard biological detection chips having the same configuration as a biological detection chip used in actual detection, to obtain corresponding detection signals, and fitting the known concentration and the corresponding detection signal to determine the dependency relationship.

In an embodiment of the present disclosure, the light includes a monochromatic light.

Further aspects and regions of applicability will become apparent from the description provided herein. It should be understood that various aspects of this disclosure may be implemented individually or in combination with one or more other aspects. It should also be understood that the description and specific examples herein are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1 is a cross-sectional schematic view of a biological detection chip in accordance with an embodiment of the present disclosure;

FIG. 2 is a flow chart of a detecting method using a biological detection chip in accordance with an embodiment of the present disclosure; and

FIGS. 3-4 are schematic views of a detecting method using a biological detection chip in accordance with an embodiment of the present disclosure.

Corresponding reference numerals indicate corresponding parts or features throughout the several views of the drawings.

### DETAILED DESCRIPTION

As used herein and in the appended claims, the singular form of a word includes the plural, and vice versa, unless the context clearly dictates otherwise. Thus, the references “a”, “an”, and “the” are generally inclusive of the plurals of the respective terms. Similarly, the words “comprise”, “comprises”, and “comprising” are to be interpreted inclusively rather than exclusively. Likewise, the terms “include”, “including” and “or” should all be construed to be inclusive, unless such a construction is clearly prohibited from the context. Where used herein the term “examples,” particularly when followed by a listing of terms is merely exemplary and illustrative, and should not be deemed to be exclusive or comprehensive.

Further to be noted, when the elements and the embodiments thereof of the present application are introduced, the articles “a/an”, “one”, “the” and “said” are intended to represent the existence of one or more elements. Unless otherwise specified, “a plurality of” means two or more. The expressions “comprise”, “include”, “contain” and “have” are intended as inclusive and mean that there may be other elements besides those listed. The terms such as “first” and “second” are used herein only for purposes of description and are not intended to indicate or imply relative importance and the order of formation.

The flow diagrams depicted herein are just one example. There may be many variations to this diagram or the steps described therein without departing from the spirit of the disclosure. For instance, the steps may be performed in a differing order or steps may be added, deleted, or modified. All of these variations are considered a part of the claimed disclosure.

Example embodiments will now be described more fully with reference to the accompanying drawings.

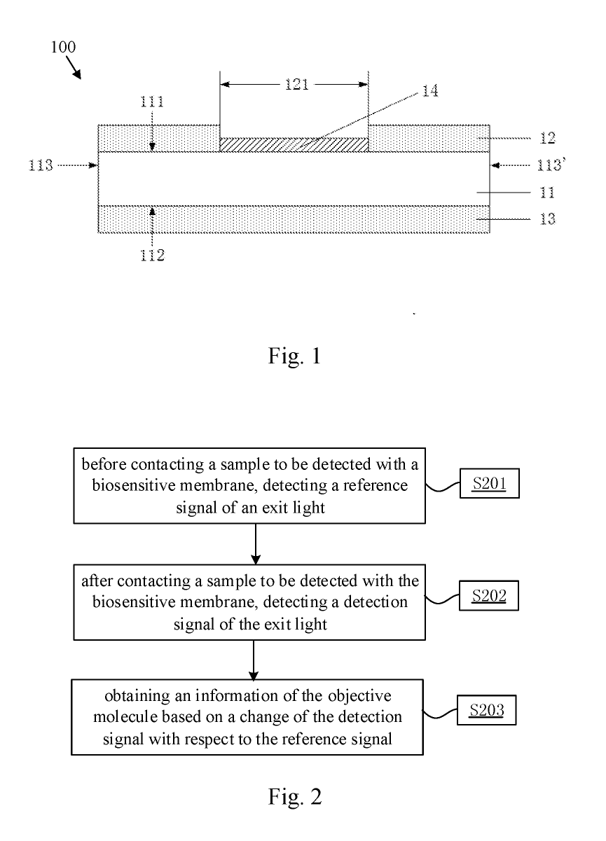

FIG. 1 is a cross-sectional schematic view of a biological detection chip in accordance with an embodiment of the present disclosure. As shown in FIG. 1, the biological detection chip 100 includes a light guiding substrate 11 and a biosensitive membrane 14. The light guiding substrate 11 has a top surface 111, a bottom surface 112 opposite to the top surface 111, and side surfaces 113, 113′ between the top surface 111 and the bottom surface 112. The biosensitive membrane 14 is located on the top surface 111 of the light guiding substrate 11. It should be noted that the biosensitive membrane 14 is capable of binding or reacting with an objective molecule in a sample to be detected (not shown), for example, specific adsorption.

Further, the biological detection chip 100 further includes a first optical confinement layer 12 on the top surface 111 of the light guiding substrate 11 and a second optical confinement layer 13 on the bottom surface 112 of the light guiding substrate 11. The first optical confinement layer 12 has an opening 121 exposing the light guiding substrate 11. The biosensitive membrane 14 is located within the opening 121.

In an exemplary embodiment of the present disclosure, in one aspect, one of the first optical confinement layer 12 and the second optical confinement layer 13 includes a light transmissive material, and the other one of the first optical confinement layer 12 and the second optical confinement layer 13 includes a reflective material or an opaque material. A refractive index of the light transmissive material is smaller than a refractive index of the light guiding substrate 11.

In an exemplary embodiment of the present disclosure, in another aspect, the first optical confinement layer 12 and the second optical confinement layer 13 include a light transmissive material. The refractive index of the light transmissive material is smaller than the refractive index of the light guiding substrate 11.

In an exemplary embodiment of the present disclosure, in still another aspect, the first optical confinement layer 12 and the second optical confinement layer 13 include a reflective material or an opaque material.

It should be understood that the relationship between the refractive index of the first and second optical confinement layers 12, 13 and the refractive index of the light guiding substrate 11 is defined, so that a light for detection incident from the side surface 113 of the light guiding substrate 11 can be totally reflected within the light guiding substrate 11, thereby the sample to be detected can be detected more accurately, and an information of the objective molecule in the sample to be detected can be obtained more accurately.

In an exemplary embodiment of the present disclosure, a material of the biosensitive membrane 14 includes one of an antibody probe, an enzyme probe, and a DNA probe. For example, in the case where it is required to detect an antibody in a sample to be detected, a biosensitive membrane including an antibody probe is used, in the case where it is required to detect an enzyme in a sample to be detected, a biosensitive membrane including an enzyme probe is used, and in the case where it is required to detect DNA in a sample to be detected, a biosensitive membrane including a DNA probe is used. The type of probe to be used can be selected according to the actual application.

In an exemplary embodiment of the present disclosure, a material of the light guiding substrate 11 includes glass or resin. It should be understood that other materials capable of propagating light may be used for the light guiding substrate 11.

In an embodiment of the present disclosure, there is also provided a detecting method using the above biological detection chip. The detecting method includes before contacting a sample to be detected with the biosensitive membrane, introducing a light for detection from one end of the light guiding substrate and detecting an exit light from the other end of the light guiding substrate to obtain a reference signal characterizing the exit light, after contacting the sample to be detected with the biosensitive membrane, detecting the exit light to obtain a detection signal characterizing the exit light and obtaining an information of an objective molecule corresponding to the biosensitive membrane in the sample to be detected based on a change of the detection signal with respect to the reference signal.

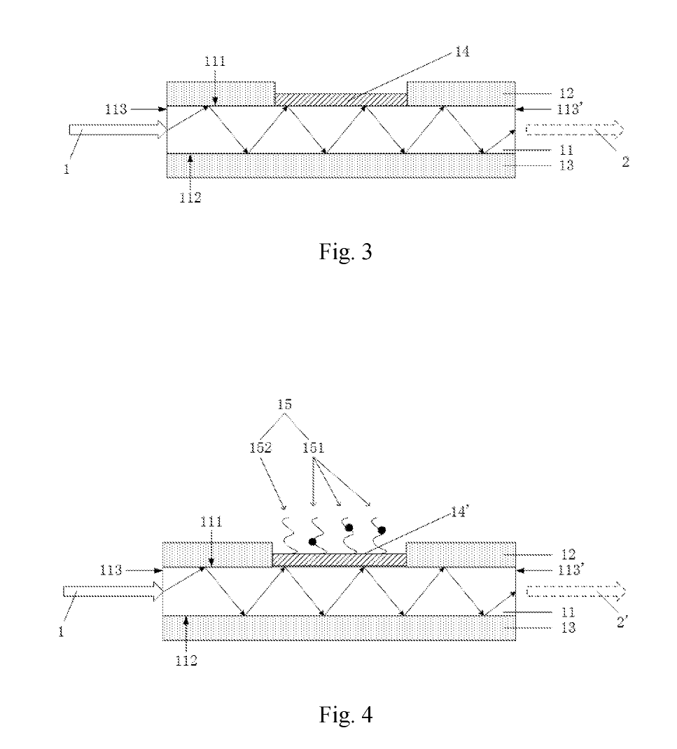

Specifically, FIG. 2 is a flow chart of a detecting method using the biological detection chip shown in FIG. 1. FIGS. 3-4 are schematic views of a detecting method using the biological detection chip shown in FIG. 1.

As shown in FIG. 2, in step S201, before contacting a sample to be detected with the biosensitive membrane, detecting a reference signal of an exit light, in step S202, after contacting a sample to be detected with the biosensitive membrane, detecting a detection signal of the exit light, and in step S203, obtaining an information of the objective molecule based on a change of the detection signal with respect to the reference signal.

As shown in FIG. 3, FIG. 3 is a schematic view showing the detection of the exit light before contacting the sample to be detected with the biosensitive membrane 14. The signal thus obtained is referred to a reference signal.

Specifically, the incident light 1 is incident into the light guiding substrate 11 from the side surface 113 of the light guiding substrate 11. The incident light 1 propagates between the top surface 111 and the bottom surface 112 of the light guiding substrate 11. When the incident light 1 is incident on the interface between the biosensitive membrane 14 and the top surface 111, the incident light 1 interacts with the biosensitive membrane 14, and then exits from the side surface 113′ of the light guiding substrate 11. The above-mentioned exit light is indicated as the exit light 2 in FIG. 3. The exit light 2 is detected by a detecting device (not shown) and a reference signal characterizing the exit light 2 is obtained. According to an embodiment of the present disclosure, the reference signal is, for example, a light power.

Subsequently, as shown in FIG. 4, FIG. 4 is a schematic view showing the detection of the exit light after the sample 15 to be detected is brought into contact with the biosensitive membrane 14. The signal thus obtained is referred to a detection signal.

In an exemplary embodiment of the present disclosure, the sample 15 to be detected is, for example, a mixture including an objective molecule 151 and a non-objective molecule 152. As an example, the objective molecule 151 can be one of an antibody, an enzyme, and DNA.

Specifically, as described above, the incident light 1 propagates inside the light guiding substrate 11, and then exits from the side surface 113′ of the light guiding substrate 11. The above-mentioned exit light is indicated as the exit light 2′ in FIG. 4. Then, the exit light 2′ is detected by a detecting device (not shown) and a detection signal is obtained. Similarly, the detection signal is the optical power.

Finally, the reference signal is compared with the detection signal, and an information of the objective molecule in the sample to be detected is obtained based on the change of the detection signal with respect to the reference signal. Specifically, when the sample to be detected is brought into contact with the biosensitive membrane, the characteristic (such as composition or thickness) of the biosensitive membrane are changed, thereby the characteristic (such as the optical power) of the exit light 2′ changes with respect to the characteristic (such as optical power) of the exit light 2, such that the detection signal changes with respect to the reference signal. Therefore, it is possible to obtain that there is objective molecule in the sample to be detected. It should be understood that if the objective molecule is not contained in the sample to be detected, the characteristic of the biosensitive membrane does not change due to the absence of adsorption of the objective molecule, and accordingly, the detection signal does not change with respect to the reference signal.

Further, according to an embodiment of the present disclosure, an information about the molecular concentration of the objective molecule in the sample to be detected can also be obtained.

Specifically, first, standard samples containing objective molecules of different known concentrations are detected, respectively, by using a plurality of standard biological detection chips having the same configuration as a biological detection chip used in actual detection, to obtain corresponding detection signals, and then fitting the known concentration and the corresponding detection signal to determine the dependency relationship between the concentration of the objective molecule and the detection signal. At the time of actual detection, based on the dependency relationship, the concentration of the objective molecule can be easily obtained by the detection signal actually obtained. It should be understood that, herein, “the same configuration” means that the structure, material, size, and the like are the same.

In an exemplary embodiment of the present disclosure, the incident light 1 may include a monochromatic light.

In the embodiment of the present disclosure, a biological detection chip and a detecting method thereof are provided. By providing a biosensitive membrane on the light guiding substrate and based on a change in the signal of the light propagating within the light guiding substrate, the information about the objective molecule in the sample can be obtained. Thus, without complex fluorescent label and optical detection systems, the micro, rapid and sensitive detection can be achieved.

The foregoing description of the embodiments has been provided for purpose of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are included within the scope of the disclosure.

1. A biological detection chip comprising:
a light guiding substrate having a top surface, a bottom surface opposite to the top surface, and a side surface between the top surface and the bottom surface; and
a biosensitive membrane on the top surface of the light guiding substrate.

2. The biological detection chip according to claim 1, further comprising a first optical confinement layer on the top surface of the light guiding substrate and a second optical confinement layer on the bottom surface of the light guiding substrate, wherein the first optical confinement layer has an opening exposing the light guiding substrate, and wherein the biosensitive membrane is located within the opening.

3. The biological detection chip according to claim 2, wherein one of the first optical confinement layer and the second optical confinement layer comprises a light transmissive material, wherein the other one of the first optical confinement layer and the second optical confinement layer comprises one of a reflective material and an opaque material, and wherein a refractive index of the light transmissive material is smaller than a refractive index of the light guiding substrate.

4. The biological detection chip according to claim 2, wherein the first optical confinement layer and the second optical confinement layer comprise a light transmissive material, and wherein a refractive index of the light transmissive material is smaller than a refractive index of the light guiding substrate.

5. The biological detection chip according to claim 2, wherein the first optical confinement layer and the second optical confinement layer comprise one of a reflective material and an opaque material.

6. The biological detection chip according to claim 1, wherein a material of the biosensitive membrane comprises one of an antibody probe, an enzyme probe, and a DNA probe.

7. The biological detection chip according to claim 1, wherein a material of the light guiding substrate comprises one of glass and resin.

8. A detecting method using the biological detection chip according to claim 1, the method comprising:
before contacting a sample to be detected with the biosensitive membrane, introducing a light for detection from one end of the light guiding substrate and detecting an exit light from the other end of the light guiding substrate to obtain a reference signal characterizing the exit light;
after contacting the sample to be detected with the biosensitive membrane, detecting the exit light to obtain a detection signal characterizing the exit light; and
obtaining an information of an objective molecule corresponding to the biosensitive membrane in the sample to be detected based on a change of the detection signal with respect to the reference signal.

9. The detecting method according to claim 8, wherein the reference signal and the detection signal comprise a light power.

10. The detecting method according to claim 8, wherein the information comprises a concentration of the objective molecule.

11. The detecting method according to claim 10, wherein the method further comprises, after obtaining the detection signal, obtaining the concentration of the objective molecule based on a dependency relationship between the detection signal obtained in advance and the concentration of the objective molecule.

12. The detecting method according to claim 11, wherein the dependency relationship is determined by the following steps:
detecting standard samples containing objective molecules of different known concentrations, respectively, by using a plurality of standard biological detection chips having the same configuration as a biological detection chip used in actual detection, to obtain corresponding detection signals; and
fitting the known concentration and the corresponding detection signal to determine the dependency relationship.

13. The detecting method according to claim 8, wherein the light comprises a monochromatic light.

14. A detecting method using the biological detection chip according to claim 2, the method comprising:
before contacting a sample to be detected with the biosensitive membrane, introducing a light for detection from one end of the light guiding substrate and detecting an exit light from the other end of the light guiding substrate to obtain a reference signal characterizing the exit light;
after contacting the sample to be detected with the biosensitive membrane, detecting the exit light to obtain a detection signal characterizing the exit light; and
obtaining an information of an objective molecule corresponding to the biosensitive membrane in the sample to be detected based on a change of the detection signal with respect to the reference signal.

15. A detecting method using the biological detection chip according to claim 3, the method comprising:
before contacting a sample to be detected with the biosensitive membrane, introducing a light for detection from one end of the light guiding substrate and detecting an exit light from the other end of the light guiding substrate to obtain a reference signal characterizing the exit light;
after contacting the sample to be detected with the biosensitive membrane, detecting the exit light to obtain a detection signal characterizing the exit light; and
obtaining an information of an objective molecule corresponding to the biosensitive membrane in the sample to be detected based on a change of the detection signal with respect to the reference signal.

16. A detecting method using the biological detection chip according to claim 4, the method comprising:
before contacting a sample to be detected with the biosensitive membrane, introducing a light for detection from one end of the light guiding substrate and detecting an exit light from the other end of the light guiding substrate to obtain a reference signal characterizing the exit light;
after contacting the sample to be detected with the biosensitive membrane, detecting the exit light to obtain a detection signal characterizing the exit light; and
obtaining an information of an objective molecule corresponding to the biosensitive membrane in the sample to be detected based on a change of the detection signal with respect to the reference signal.

17. A detecting method using the biological detection chip according to claim 5, the method comprising:
before contacting a sample to be detected with the biosensitive membrane, introducing a light for detection from one end of the light guiding substrate and detecting an exit light from the other end of the light guiding substrate to obtain a reference signal characterizing the exit light;
after contacting the sample to be detected with the biosensitive membrane, detecting the exit light to obtain a detection signal characterizing the exit light; and
obtaining an information of an objective molecule corresponding to the biosensitive membrane in the sample to be detected based on a change of the detection signal with respect to the reference signal.

18. A detecting method using the biological detection chip according to claim 6, the method comprising:
before contacting a sample to be detected with the biosensitive membrane, introducing a light for detection from one end of the light guiding substrate and detecting an exit light from the other end of the light guiding substrate to obtain a reference signal characterizing the exit light;
after contacting the sample to be detected with the biosensitive membrane, detecting the exit light to obtain a detection signal characterizing the exit light; and
obtaining an information of an objective molecule corresponding to the biosensitive membrane in the sample to be detected based on a change of the detection signal with respect to the reference signal.

19. A detecting method using the biological detection chip according to claim 7, the method comprising:
before contacting a sample to be detected with the biosensitive membrane, introducing a light for detection from one end of the light guiding substrate and detecting an exit light from the other end of the light guiding substrate to obtain a reference signal characterizing the exit light;
after contacting the sample to be detected with the biosensitive membrane, detecting the exit light to obtain a detection signal characterizing the exit light; and
obtaining an information of an objective molecule corresponding to the biosensitive membrane in the sample to be detected based on a change of the detection signal with respect to the reference signal.
